SEQUENCE LISTING (3 sequences)

SEQ ID NO: 1 - protein, 9 aa, Artificial Sequence
  Artificial Sequence = synthetic construct
KRSYEEHIP

SEQ ID NO: 2 - protein, 16 aa, Artificial Sequence
  Artificial Sequence = synthetic construct
VLPQDKEYYKVKEPGE

SEQ ID NO: 3 - protein, 12 aa, Artificial Sequence
  Artificial Sequence = synthetic construct
TRDIYETDYYRK

(19) United States
(12) Patent Application Publication
(10) Pub. No.: US 2009/0142849 A1
(43) Pub. Date: Jun. 4, 2009

(54) Stable Oxygen Isotope Labeling of Pre-existing Phosphoryl Groups on Phosphomolecules for Modification-Specific Mass Spectrometry

(75) Inventor: Xudong Yao, Storrs, CT (US)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: University of Connecticut, Farmington, CT (US)

(21) Appl. No.: 12/245,260

(22) Filed: Oct. 3, 2008

Related U.S. Application Data
(60) Provisional application No. 60/977,774, filed on Oct. 5, 2007.

(51) Int. Cl. G01N 33/68

(52) U.S. Cl. 436/86; 436/104

(57) ABSTRACT

The present invention relates to the production and use of phosphate-specific marker ions labeled with one or more stable oxygen isotopes for analysis of phosphopeptides and other phosphorylated biological and synthetic molecules.

### CROSS-REFERENCE TO RELATED APPLICATIONS

This application is a continuation-in-part of and claims benefit of priority from U.S. Provisional Application No. 60/977,774, filed Oct. 5, 2007, which is herein incorporated by reference in its entirety.

### FIELD OF THE INVENTION

The present invention relates to the production and use of phosphate-specific marker ions labeled with one or more stable oxygen isotopes for mass spectrometry analysis of phosphopeptides and other phosphorylated biological and synthetic molecules.

### BACKGROUND

Phosphorylation and de-phosphorylation of polypeptides and other biological molecules is one important way in which cellular activities are regulated. The net protein phosphorylation state in a cell results from the delicate balance between enzymatic reactions catalyzed by protein kinases and phosphatases in the cell. These enzymes add or remove phosphate groups on polypeptides. Other biological molecules can undergo analogous phosphorylation and de-phosphorylation reactions. Approximately one-third of the proteins in a eukaryotic cell are phosphorylated at any given time. These phosphoproteins comprise the phosphoproteome.

The ability to accurately measure both the relative phosphorylation level at individual phosphorylation sites and the number of phosphorylated sites on proteins is essential to understanding cellular phosphorylation events in quantitative and time-resolved manners. Phosphoproteome analysis has benefited from advances in mass spectrometry and been further facilitated by stable isotope labeling of phosphoproteins and phosphopeptides. Various types of labeling strategies are known in the art.

One previously known labeling method involves isotopically labeling phosphopeptides at sites that differ from the phosphopeptide phosphorylation sites. After combining differentially labeled samples, phosphopolypeptides are often subjected to affinity enrichment for quantitative phosphoproteome analysis. Mass spectrometric signals for the differentially-labeled counterpart peptides are separated by a defined mass that is equal to the mass difference between different isotope labels. The relative intensities of the signals for the counterpart peptides can be used as the measure of their relative concentrations. Furthermore, if the amount of one of the peptides is known, the absolute quantity of the counterpart peptide can be calculated (Gerber et al., Proc Natl Acad Sci USA 2003, 100, (12), 6940-5; Mayya et al., Mol Cell Proteomics 2006, 5, (6), 1146-1157). Common stable isotope labeling methods, including chemical (Gygi et al., Nat Biotechnol 1999, 17, (10), 994-9; Chakraborty and Regnier, J Chromatogr A 2002, 949, (1-2), 173-84), metabolic (Oda et al., Proc Natl Acad Sci USA 1999, 96, (12), 6591-6; 4; Chen et al., Anal Chem 2000, 72, (6), 1134-43; Ong et al., Mol Cell Proteomics 2002, 1, (5), 376-86) and enzymatic labeling (Yao et al., Anal Chem 2001, 73, (13), 2836-42), have been used for mass spectrometry analysis.

A second type of labeling strategy involves chemical derivatization of peptidyl phosphates to introduce chemical tags directly at phosphorylation sites of proteins. For example, serinyl and threoninyl phosphates undergo beta-elimination reactions under basic conditions to generate polarized carbon-carbon double bonds that can be further derivatized by bifunctional chemical tags. The tag molecules have a nucleophile that can react with the newly generated double bond, as well as a second chemical structure such as an isotope label (see, e.g., Goshe et al., Anal Chem 2002, 74, (3), 607-16; Glinski et al., Rapid Commun in Mass Spec 2003, 17, (14), 1579-1584; Amoresano et al., Euro J Mass Spec 2004, 10, (3), 401-412) that can help in the preparation, separation, detection, or quantitation of the tagged phosphopeptides.

Another type of chemistry used to directly tag phosphorylation sites involves carbodiimide-assisted amidation of peptidyl phosphates (Sullivan et al., Anal. Biochem. 1991, 197, (1), 65-8). In contrast to the beta-elimination-based derivatization of phosphoserinyl and phosphothreoninyl groups, this method is universal to phosphate groups on biomolecules including phosphoserinyl (pS), phosphothreoninyl (PT), and phosphotyrosinyl (pY) peptides, and derivatization products do not exist as diastereomers. However, in this approach peptidyl amines must be protected or a large excess of modifying amines need to be introduced to prevent the side reactions of peptide-peptide coupling. Moreover, carbodiimide-assisted phosphate derivatization requires a step of methanolic HCl esterification of the carboxylate to minimize the competing reaction of carboxylate groups on peptides.

A major limitation of beta-elimination-based and carbodiimide-assisted derivatizations of peptidyl phosphates is that these chemical reactions alter peptidyl phosphates into a final chemical structure that is different from the original chemical structure. This alteration changes or eliminates gaseous marker ions from the phosphates, prohibiting the direct application of phosphate-specific mass spectrometry approaches.

Phosphate-specific ions have been used to develop mass spectrometric methods for the selective identification of phosphopeptides and to pinpoint sites that can be phosphorylated within specific polypeptides of interest (Carr et al., Methods Enzymol 2005, 405, 82-115; Beck, Rapid Commun Mass Spectrom 2001, 15, (23), 2324-33; Steen et al., Analyt Chem 2001, 73, (7), 1440-1448.). Phosphopeptides generate phosphate-specific marker ions of m/z 63.964 (PO₂⁻), 78.959 (PO₃⁻), and 96.969 (H₂PO₄⁻) in negative ion mode. Peptides with phosphorylated tyrosine (pY) residues produce a characteristic positive immonium ion of m/z 216.043. These phosphate-specific ions have allowed the development of several tandem mass spectrometry (MS/MS) methods such as precursor scan, neutral loss scan, and precursor ion discovery.

Recently, another method for detecting protein phosphorylation sites by mass spectrometry has been reported (Zhou et al., Analytical Chemistry 2007, 79, (20), 7603-7610). In this approach, proteins were phosphorylated by an in vitro kinase reaction using adenosine 5′-triphosphate-γ-¹⁸O₄ (ATP-γ-¹⁸O₄) as the phosphoryl donor. The newly-added protein phosphate group carried three ¹⁸O atoms with an incremental mass of 6.012 Da, relative to the ¹⁶O-labeled phosphate. Analysis of the ¹⁸O-labeled proteins by mass spectrometry allows the detection of protein amino acid residues that are capable of being phosphorylated by kinases. However, this approach does not allow the direct labeling of the non-bridging oxygen atoms on pre-existing phosphoryl groups (i.e., those phosphoryl groups that result from in vivo phosphorylation). Thus, this method does not allow the direct detection of pre-existing phosphoryl groups on a phosphoprotein or other phosphorylated molecule.

The direct labeling of the nonbridging oxygen atoms on pre-existing phosphate groups on polypeptides, such as those present within in vivo-phosphorylated protein samples, remains an unsolved challenge for the preparation of labeled phosphopeptide samples for mass spectrometry. Thus, there is a need in the art for methods and compositions for directly labeling pre-existing phosphopeptides and other phosphorylated biological and synthetic molecules, and for identifying and quantifying such molecules using mass spectrometric approaches.

### SUMMARY

Phosphorylation and de-phosphorylation of polypeptides and other biological molecules is one important way in which cellular activities are regulated. The ability to accurately measure both the relative phosphorylation level at individual phosphorylation sites and the number of phosphorylated sites on proteins is essential to understanding cellular phosphorylation events. However, methods for detecting the presence of pre-existing phosphoryl groups without changing the pre-existing structure of molecules carrying the phosphoryl groups have been lacking. The invention provides compositions, methods, kits, and software for detecting such phosphoryl groups without changing the pre-existing structure of the molecules to which the phosphoryl groups are attached.

In a first aspect, the invention provides a method of labeling a phosphomolecule with a stable oxygen isotope. The method can include the steps of: a) activating a phosphoryl group covalently bound to the phosphomolecule, and b) hydrolyzing the activated phosphoryl group with water enriched for a stable oxygen isotope, thereby labeling the phosphomolecule with the stable oxygen isotope.

In one embodiment the activated phosphoryl group can be amidated prior to hydrolyzing the activated phosphoryl group with water enriched for a stable oxygen isotope.

In another embodiment the phosphomolecule can be a phosphopeptide.

In yet another embodiment the stable oxygen isotope can be ¹⁶O, ¹⁷O, or ¹⁸O.

In a second aspect, the invention provides a kit for labeling a phosphoryl group on a phosphomolecule with a stable oxygen isotope. The kit can include: a) a reagent for activating the phosphoryl group covalently bound to the phosphomolecule; b) a reagent for donating an amine group to form an amide bond with the phosphorus of a phosphate group that is covalently bound to the phosphomolecule; and c) instructions for using the kit to label a phosphoryl group on a phosphomolecule with a stable oxygen isotope.

In one embodiment the kit can include water that is enriched for a stable oxygen isotope, e.g., ¹⁶O, ¹⁷O, or ¹⁸O.

In another embodiment the kit can include a reagent for methylating carboxyl residues of the phosphomolecule prior to activating the phosphoryl group covalently bound to the phosphomolecule.

In still another embodiment the kit can include a control phosphomolecule.

In yet another embodiment the kit can include a material for purifying a labeled phosphomolecule from a reaction mixture.

In a third aspect, the invention provides a kit for labeling a phosphoryl group on a phosphomolecule with a stable oxygen isotope. The kit can include: a) a reagent for activating the phosphoryl group covalently bound to the phosphomolecule; b) a reagent for donating a functional group to form an active ester with a non-bridging oxygen of a phosphate group that is covalently bound to the phosphomolecule; and c) instructions for using the kit to label a phosphoryl group on a phosphomolecule with a stable oxygen isotope.

In one embodiment the kit can include water that is enriched for a stable oxygen isotope.

In another embodiment, the kit can include a reagent for methylating carboxyl residues of the phosphomolecule prior to activating the phosphoryl group covalently bound to the phosphomolecule.

In still another embodiment the kit can include a control phosphomolecule.

In yet another embodiment the kit can include a material for purifying a labeled phosphomolecule from a reaction mixture.

In a fourth aspect, the invention provides a composition comprising a phosphate-specific marker ion. The phosphate-specific marker ion is labeled with a stable oxygen isotope and the phosphate-specific marker ion labeled with the stable oxygen isotope is present at a higher concentration than would be present in a naturally-occurring sample containing the phosphate-specific marker ion.

In one embodiment the phosphate-specific marker ion is Po₂⁻, PO₃⁻, H₂PO₄⁻, or immonium ion of phosphotyrosine.

In another embodiment the stable oxygen isotope is ¹⁶O, ¹⁷O, or ¹⁸O.

In yet another embodiment the phosphate-specific marker ion is labeled at only one non-bridging oxygen position within the phosphate specific marker ion.

In still another embodiment the phosphate-specific marker ion is labeled at only two non-bridging oxygen positions within the phosphate specific marker ion.

In a yet another embodiment the phosphate-specific marker ion is labeled at all three non-bridging oxygen positions within the phosphate specific marker ion.

In a fifth aspect, the invention provides a phosphate-specific marker ion comprising at least one non-bridging oxygen atom position labeled with a stable oxygen isotope.

In one embodiment the phosphate-specific marker ion further comprises a bridging oxygen atom position not labeled with a stable oxygen isotope.

In a sixth aspect, the invention provides a phosphomolecule carrying a covalently-bound phosphoryl group, wherein the phosphoryl group has two or fewer non-bridging oxygen atom positions labeled with a stable oxygen isotope.

In one embodiment the phosphoryl group has only one non-bridging oxygen atom position labeled with a stable oxygen isotope.

In another embodiment the phosphoryl group has a bridging oxygen atom position not labeled with a stable oxygen isotope.

In still another embodiment the phosphomolecule is a phosphopeptide. The phosphoryl group of the phosphopeptide can be covalently bound to tyrosine, serine, or threonine.

In a seventh aspect, the invention provides a method of detecting a pre-existing phosphoryl group on a phosphomolecule. The method can include: a) subjecting a phosphomolecule labeled with a stable oxygen isotope to a gas-phase dissociation method to generate a labeled phosphate-specific marker ion, wherein the phosphoryl group has at least one non-bridging oxygen atom position labeled with a stable oxygen isotope; and b) detecting an ion signal produced by the labeled marker ion, thereby detecting the pre-existing phosphoryl group on the phosphomolecule.

In one embodiment the phosphoryl group can have a bridging oxygen atom position not labeled with a stable oxygen isotope.

In an eighth aspect, the invention provides a method of measuring the number of pre-existing phosphoryl groups on a phosphomolecule in a sample. The method can include: a) subjecting a labeled phosphomolecule from a first sample to a gas-phase dissociation method to generate a labeled phosphate-specific marker ion, wherein the phosphoryl group in the labeled phosphomolecule has at least one non-bridging oxygen atom position labeled with a stable oxygen isotope; b) measuring an ion signal produced by the labeled phosphate-specific marker ion; and c) comparing the ion signal from the labeled phosphate-specific marker ion from the first sample to an ion signal produced by a labeled phosphate-specific marker ion from the labeled phosphomolecule from a second sample, wherein the labeled phosphomolecule from the second sample has a different number of non-bridging oxygen atom positions labeled with a stable oxygen isotope than does the labeled phosphomolecule from the first sample, thereby measuring the number of pre-existing phosphoryl groups on a phosphomolecule in a sample.

In one embodiment the labeled phosphomolecule from the first and second samples can be combined into a single sample prior to subjecting the phosphomolecule to a gas-phase dissociation method.

In another embodiment the labeled phosphomolecule from a third sample can be combined with the labeled phosphomolecule from the first and second samples prior to subjecting the phosphomolecule to a gas-phase dissociation method, and ion signals produced from all three samples can be compared. The labeled phosphomolecule from the third sample has a different number of oxygen atom positions labeled with a stable oxygen isotope than does the labeled phosphomolecule from the first and second samples.

In still another embodiment the labeled phosphomolecule from a fourth sample can be combined with the labeled phosphomolecule from the first, second, and third samples prior to subjecting the phosphomolecule to a gas-phase dissociation method, and ion signals produced from all four samples are compared. The labeled phosphomolecule from the fourth sample has a different number of oxygen atom positions labeled with a stable oxygen isotope than does the labeled phosphomolecule from the first, second, and third samples.

In yet another embodiment the phosphoryl group has a bridging oxygen atom position not labeled with a stable oxygen isotope.

In a ninth aspect, the invention features a method of measuring the relative concentration of a pre-existing phosphoryl group on a phosphomolecule in two samples. The method can include the steps of: a) combining together a first sample with a second sample to produce a single sample, wherein the phosphoryl group in the first sample is unlabeled and the phosphoryl group in the second sample is labeled with ¹⁷O or ¹⁸O; and b) comparing an ion signal produced by the unlabeled phosphate-specific marker ion with an ion signal produced by the labeled phosphate-specific marker ion, thereby measuring the relative concentration of a pre-existing phosphoryl group on a phosphomolecule in the two samples.

In a tenth aspect, the invention features software for controlling a mass spectrometer. Simultaneous detection by the mass spectrometer of an unlabeled marker ion plus a labeled marker ion or simultaneous detection of two differentially-labeled marker ions causes the software to instruct the mass spectrometer to search for precursor ions for the differentially-labeled ions, and detection of the precursor ions causes the software to instruct the mass spectrometer to target the precursor ions for fragmentation to obtain structural information.

In an eleventh aspect, the invention features software for controlling a mass spectrometer. The software, upon receiving a signal that the mass spectrometer has detected a neutral loss of H₃PO₄ or HPO₃ from a phosphorylated molecule, instructs the mass spectrometer to search for targeted fragmentation of a precursor molecule for the neutral loss. The H₃PO₄ or HPO₃ can be singly or doubly labeled with a stable oxygen isotope.

In a twelfth aspect, the invention features data-processing software that uses marker ions for the identification or quantitation of a phosphorylated molecule. The software uses relative intensities of differentially-labeled phosphate-specific marker ions to quantify precursor molecules of the differentially-labeled phosphate-specific marker ions.

In a thirteenth aspect, the invention features data-processing software that uses differentially-labeled phosphorylated molecules for confirming the presence of a phosphoryl group and determining the number of phosphoryl groups on a phosphorylated molecule. The software uses mass difference between differentially-labeled phosphorylated molecules and marker ions from the differentially-labeled phosphorylated molecules to determine the number of phosphoryl groups.

In a fourteenth aspect, the invention features data-processing software that uses a neutral loss of H₃PO₄ or HPO₃ from a phosphorylated molecule to identify and confirm a phosphorylation site of the phosphorylated molecule. The H₃PO₄ or HPO₃ from the phosphorylated molecule can be singly or doubly labeled with a stable oxygen isotope.

### BRIEF DESCRIPTION OF THE DRAWINGS

FIG. 1. FIG. 1A depicts a phosphopeptide; the substructure in the open box represents peptidyl phosphate; letter “a” denotes nonbridging oxygen atoms. FIG. 1B denotes a derivatized phosphopeptide whose phosphate group is converted to a phosphoramidate (in the open box); the P—N bond is a phosphoramide bond. FIG. 1C represents a phosphopeptide with a phosphate-¹⁸O₁. FIG. 1D represents a phosphopeptide with a phosphate-¹⁶O₁.

FIG. 2. Phosphoramidate-mediated oxygen labeling of peptidyl phosphates. The reaction schematic illustrates the phosphoramidate-mediated exchange reaction of one peptidyl phosphate oxygen with water. Carboxylate groups on the phosphopeptide are permethylated. When H₂¹⁸O is used, the phosphate incorporates one ¹⁸O atom; when H₂¹⁶O is used, the phosphate incorporates one ¹⁶O atom. Spectrum A is for the phosphate-¹⁸O₁ of NPF peptide. Spectrum B is for NPF peptidyl phosphate-¹⁶O₁.

FIG. 3. Isotopic substitution of oxygen atoms on three phosphate groups of a phosphopeptide. The upper spectrum is for the insulin receptor (5) phosphopeptide with three ¹⁶O₁-labeled phosphates. The lower spectrum is for the insulin receptor (5) phosphopeptide after ¹⁸O substitution; the most abundant peak (I₆) corresponds to the mass-to-charge ratio of the peptide with three phosphates-¹⁸O₁.

FIG. 4. Pseudo first order kinetics for hydrolysis of NPF peptidyl phosphoramidate. NPF peptidyl phosphate-¹⁸O₁ was used as the internal standard. Section A shows the mass spectra for the peptidyl phosphate in the reaction mixtures at different time (from time 0 to overnight). Section B illustrates that the peptidyl phosphate-¹⁸O₁ is stable under the hydrolysis condition (rounded dots) and R_{corr}^{t} is the corrected ratios of the phosphate-¹⁶O₁ to phosphate-¹⁸O₁. Section C is the linear plot for the pseudo first order hydrolysis reaction.

FIG. 5. p² CID mass spectrum of the NPF phosphopeptide. Insert A magnifies the region for the pY immonium ions. Insert B magnifies the region for the intact singly charged molecular ions. The correlation plot suggests the authentic sampling in quantitative information of the phosphopeptide by the marker ions.

FIG. 6. Sequence-independent quantitation of pY peptides using phosphate-specific marker ions and p²CID-MS.

FIG. 7. Unique mass defect of phosphate-specific marker ions. This is a centroid mass spectrum for illustrating the concept that the unique mass defect of the pY immonium ions can be used to improve the specificity of mass spectrometric quantitation.

FIG. 8. Use of the differential ¹⁶O₁ and ¹⁸O₁-labeling as molecular ruler for mass spectrometric counting of the number of phosphate groups. A is for KRSpYEEHIP (SEQ ID NO: 1), B is for VLPQDKEpYpYKVKEPGE (SEQ ID NO: 2), and C is for TRDIpYETDpYpYRK (SEQ ID NO: 3).

### DETAILED DESCRIPTION

The activity of many important biological molecules is regulated by altering phosphorylation status of the molecules, which in turn regulates cellular activity. Thus, the ability to detect and measure phosphorylation status of important biological molecules is crucial to understanding the regulation of cellular activity. Mass spectrometry provides a sensitive approach for determining relative and absolute quantities of one or more phosphorylated molecules and for determining phosphorylation status. Phosphoproteins (i.e., proteins having one or more pre-existing phosphate groups) are one very important category of phosphorylated biological molecules. However, a serious shortcoming in the art has been a lack of methods for labeling and analyzing phosphopeptides from phosphoproteins without altering the pre-existing chemical structure of the phosphopeptides. Such a deficit prevents the accurate analysis of phosphorylation status of phosphoproteins and other phosphomolecules.

The present invention provides methods for labeling pre-existing phosphates on phosphomolecules with a stable oxygen isotope at one or more non-bridging oxygen atom positions. The stable oxygen isotopes to be used in the methods of the invention are ¹⁶O, ¹⁷O, ¹⁸O.

Phosphoryl groups bound to phosphopeptides or other phosphorylated molecules contain three non-bridging oxygen atoms (and one bridging oxygen atom) bound to the phosphorus atom. Using a peptidyl phosphate labeled with ¹⁸O as an example, the peptidyl phosphate labeled at the non-bridging oxygen atom positions can have four different labeling states, i.e., either zero, one, two, or three atoms of ¹⁸O bound to phosphorus. Direct labeling of pre-existing phosphates on peptides or other phosphorylated molecules (whether naturally-occurring or synthetic) allows the use of phosphate-specific marker ions for counting the phosphorylation number (i.e., the number of pre-existing phosphoryl residues) on proteins on other phosphorylated molecules, without altering the native structure of the molecule. The direct labeling methods and compositions of the invention also allows for detection, identification, or quantification of phosphopeptides and other naturally-occurring and synthetic phosphorylated molecules. The methods and compositions of the invention can be used to determine the phosphorylation number (i.e., the number of phosphorylated positions on the molecule) and relative changes in phosphorylation number between or among different samples containing a particular phosphomolecule to be analyzed.

Under uncatalyzed or mild conditions, peptidyl phosphates are resistant to exchanging their non-bridging oxygen atoms with water; thus, they are difficult to label with oxygen isotope labels. Prior art methods have employed enzyme catalysis or chemical reactions under extreme conditions to label inorganic phosphate with ¹⁸O via an exchange with H₂¹⁸O. However, such approaches cannot be used to exchange the nonbridging oxygen atoms (the three oxygen atoms that are not bound to an amino acid residue) on peptidyl phosphates without loss of the phosphoryl group or without significant degradation of the peptide structure.

The present invention expands the usefulness of the established phosphate-specific marker ions by allowing the direct isotopic substitution of non-bridging phosphate ¹⁶O atoms with ¹⁸O atoms (or ¹⁷O atoms) in phosphopeptides or other phosphorylated molecules, whether naturally-occurring or synthetic. The isotopic labeling approach described herein facilitates mass spectrometry analysis for quantifying the phosphorylation level and for counting the phosphorylation number of a phosphopeptide or other phosphorylated molecule without chemically altering the pre-existing protein structure.

The invention also provides phosphate-specific oxygen isotope-labeled marker ions that allow the sequence-independent quantitation of phosphopeptides and other phosphorylated molecules using mass spectrometry approaches such as tandem parallel collision-induced dissociation mass spectrometry (p²CID-MS) (Ramos, A. A. et al., Tandem Parallel Fragmentation of Peptides for Mass Spectrometry. Analytical Chemistry 2006, 78, (18), 6391-6397). The present invention also provides methods for using differentially-labeled oxygen isotope phosphopeptides or other phosphorylated molecules to improve the confidence levels in mass spectrometry measurements of phosphopeptide other phosphorylated molecules. The differentially-labeled phosphomolecules of the invention can also be used to perform multiplex mass spectrometry measurements, e.g., to compare phosphopeptide levels in two or more samples.

The methods and compositions of the invention can be used with phosphorylated molecules of biological or synthetic origin. Such molecules include phosphopeptides, phosphoproteins, phosphosaccharides, phosphopolysaccharides, phospholipids, phosphorylated nucleic acids such as deoxyribonucleic acid (DNA) and ribonucleic acid (RNA), phosphorylated metabolites, and synthetic phosphorylated molecules.

### Labeling.

Carbodiimide-mediated exchange reactions are used in the methods of the invention to incorporate up to three ¹⁸O (or ¹⁷O) atoms into peptidyl phosphate groups or the phosphate groups of other phosphorylated molecules. Carbodiimide activates peptidyl phosphates to form isourea intermediates. These intermediates can react with amines and convert phosphates into phosphoramidates. One convenient source of amine groups for use in the methods of the invention is propargylamine, although the skilled artisan will understand that any source of amines that has a primary or secondary amino group can be used, e.g., but not limited to, alkyl amines, aromatic amines, and heterocyclic compounds that have at least one primary or secondary amino group.

Hydrolysis of the phosphoramides introduces one hydroxyl group from water into each phosphorus atom and re-generates each phosphate group. When H₂¹⁸O is used for the hydrolysis reaction, the newly restored phosphate groups carry one label of ¹⁸O atom. Similarly, when H₂¹⁶O or H₂¹⁷O is used for the hydrolysis reaction, the newly restored phosphates carry one label of ¹⁶O atom or ¹⁷O atom, respectively.

EDC is representative of an entire class of carbodiimide compounds that can be used for the labeling reaction. However, any molecule that can activate phosphate groups to, directly or indirectly, form reactive intermediates that can be hydrolyzed to yield the labeled phosphate-specific markers of the invention may be used.

All of the activating reagents have a common characteristic application; they activate a carboxylate group to be coupled directly with an amino group, or to be trapped by a third compound (Albericio F. Curr Opin Chem. Biol. 2004, 8, 211-21). The trapped activated carboxylate group can react subsequently with an amino group to form an amide bond, or with water to regenerate carboxylate group. These reactions can also be performed on phosphate groups. Activation reagents for a phosphate group include, but not limited to, e.g., carbodiimides, onium salts, anhydrides, and acid fluorides, e.g., benzotriazolyloxytris(dimethylamino)phosphonium hexafluorophosphate, O-(7-azabenzotriazol-1-yl)-tris(dimethylamino)phosphonium, diphenylphosphorylazide (Albericio F. Curr Opin Chem. Biol. 2004, 8, 211-21).

Another class of activation reagents that can be used in the labeling methods of the invention includes organophosphorus esters such as diethoxyphosphinyloxy-7-azabenzotriazole and diethoxyphosphinyloxybenzotriazole (Albericio F. Curr Opin Chem. Biol. 2004, 8, 211-21).

Another activation system uses oxidative activation of phosphate groups, using triphenyl phosphine and dipyridyl disulfides. The activated phosphate groups can either react with amino compounds to form phosphoramidates, or react with trapping reagents such as 1-hydroxybenzotriazole, 1-hydroxy-7-azabenzotriazole, or 1-hydroxy-5-chlorobenzotriazole. The trapped phosphate groups can be hydrolyzed directly in oxygen isotope-enriched water to introduce one oxygen isotope label, or to react with an amino compound, followed by the hydrolysis to label one non-bridging phosphate oxygen.

Trapping reagents that can react with activated phosphate groups to form active esters for hydrolysis to introduce an oxygen label, or formation of phosphoramidates include, but are not limited to, e.g., 1-hydroxybenzotriazole, 1-hydroxy-7-azabenzotriazole, and 1-hydroxy-5-chlorobenzotriazole.

The isourea intermediates can also be hydrolyzed directly by water to introduce an oxygen label, without first reacting the isourea intermediates with amines to convert the phosphate groups into phosphoramidates or other hydrolyzable phosphate derivatives. When the activation-hydrolysis process in situ occurs multiple times, the ultimate reaction products carry three oxygen labels, ¹⁸O₃, ¹⁷O, or ¹⁶O₃. Multiple portions of carbodiimide can be introduced into the reaction mixture to improve the exchange efficiency.

For example, exchange of three non-bridging oxygen atoms with water oxygen can be achieved by repetitive hydrolysis of activated phosphate groups. Methylated phosphopeptides can be incubated with excess amount of 1-(3-dimethylaminopropyl)-3-ethylcarbodiimide (EDC) hydrochloride in solutions that are made with enriched H₂¹⁸O. Multiple portions of EDC can be added to increase the efficiency of the reaction. EDC or another activating molecule is used to form reactive intermediates of phosphate groups on peptides. EDC is an efficient and convenient activating molecule for use in the labeling methods of the invention. EDC represents a whole class of carbodiimide compounds that can be used for the labeling reaction. However, any molecule that activates phosphate groups in aqueous solution to form reactive intermediates that can be in situ-hydrolyzed to yield the labeled phosphate-specific markers of the invention may be used. Examples of such activating molecules include, but are not limited to, e.g., onium salts like 2-(5-norbornene-2,3-dicarboximido)-1,1,3,3-tetramethyluronium tetrafluoroborate and 4-(4,6-dimethoxy-1,3,5-triazin-2-yl)-4-methylmorpholinium chloride. Hydrolysis of the activated phosphates results in isotopic substitution of phosphate oxygen. One reaction cycle results in one oxygen exchange. Repetition of the activation-hydrolysis exchanges all of the three ¹⁸O atoms.

To introduce two atoms of isotopic oxygen into the labeled phosphate group, phosphoryl groups on phosphomolecules (e.g., phosphopeptides) are first activated with carbodiimide as described above and then modified into phosphodiamides, using compounds that have at least two primary or secondary amino groups. Another way to produce a phosphate group with a double oxygen isotope label is to exchange all three oxygen atoms as described above and then exchange one back to the original oxygen isotope (e.g., introduce three ¹⁸O labels and then exchange one ¹⁸O label for ¹⁶O, resulting in a molecule with two ¹⁸O labels).

Thus, as described above, the labeling methods of the invention can be used to introduce either one, two, or three atoms of oxygen into a pre-existing phosphate group on a peptide or other phosphomolecule. The ability to generate four different labeling states can be used for multiplex analysis, as described herein.

### DEFINITIONS

In this specification and in the claims that follow, reference is made to a number of terms that shall be defined to have the following meanings.

As used in the specification and in the appended claims, the singular forms “a,” “an,” and “the” include plural referents unless the context clearly dictates otherwise. For example, “a molecule” can mean a single molecule or more than one molecule.

“Stable oxygen isotope” means a non-radioactive isotope of oxygen, such as ¹⁶O, ¹⁷O, or ¹⁸O.

“Phosphate-specific marker ion” means an ion, such as PO₃⁻, PO₂⁻, H₂PO₄⁻, or immonium ion of phosphotyrosine, that can be produced by gaseous fragmentation (also known as gas-phase dissociation) methods such as collision induced dissociation (CID) to detect, identify, quantitate, or analyze a phosphomolecule of interest, as will be understood by one of ordinary skill in the art and as described herein.

“Labeled” means a phosphate-specific marker ion or phosphomolecule that has been subjected to an oxygen isotope exchange reaction according to the methods of the invention such that the marker ion or phosphomolecule is enriched for a stable oxygen isotope, i.e., contains a larger number of stable oxygen isotope atoms than would be expected in a sample not labeled with the stable oxygen isotope.

“Enriched” means that a stable oxygen isotope such as ¹⁶O, ¹⁷O, or ¹⁸O is present in a sample at a higher concentration than expected. By way of example, a natural sample of water would contain about 0.2% ¹⁸O. Thus, a sample that is enriched for ¹⁸O (i.e., H₂¹⁸O) contains a higher concentration of H₂¹⁸O than would be found in a natural water sample. As is understood by the skilled artisan, such water preparations enriched for stable oxygen isotopes are commercially available, as described herein.

“Phosphomolecule” means a naturally-occurring or synthetic molecule, such as a phosphopeptide, phosphopolypeptides, phosphoprotein, phospholipid, phosphosaccharide, phosphorylated metabolite, or any other molecule that contains at least one covalently-attached phosphoryl moiety. Inorganic phosphate is specifically excluded from the definition of “phosphomolecule.”

“Pre-existing phosphoryl group” or “pre-existing phosphate group” or similar language means that a phosphomolecule of interest contains a covalently-bonded phosphoryl group prior to subjecting the phosphomolecule to an oxygen isotope labeling process, e.g., an oxygen isotope labeling process described herein.

Kits for Labeling Phosphomolecules with Stable Oxygen Isotopes.

The present invention provides kits for labeling phosphomolecules with stable oxygen isotopes such as ¹⁶O, ¹⁷O, or ¹⁸O. Each kit will contain the following components: 1) a reagent for activating the phosphoryl group covalently bound to the phosphomolecule, 2) a reagent for donating an amine group to form an amide bond with the phosphorus of a phosphate group that is covalently bound to the phosphomolecule, and 3) instructions for using the kit to label a phosphoryl group on a phosphomolecule with a stable oxygen isotope.

The kits can optionally contain a control phosphorylated molecule, such as a phosphopeptide or other phosphorylated molecule.

The kits of the invention may optionally include, or be used in combination with, H₂O enriched for H₂¹⁸O, or H₂ ¹⁷O, such that the stable oxygen isotope is present at a higher concentration than would be present in a naturally-occurring water sample. For example, water containing greater than 95% ¹⁸O or greater than 97% ¹⁸O can be purchased from Sigma-Aldrich (St. Louis, Mo.) or Spectra Stable Isotopes (Columbia, Md.). By way of example, ¹⁶O is by far the most abundant isotope on the planet. In contrast, the stable oxygen isotope ¹⁸O typically represents only 0.2% of oxygen in water and thus in phosphomolecules such as phosphoproteins and phosphopeptides. Thus, the methods of the invention mass spectrometry can be performed using phosphomolecules labeled by the methods of the invention such that the phosphomolecules have been enriched for a stable oxygen isotope relative to an unlabeled sample. Typically the enrichment is at least: 2%, 5%, 10%, 20%, 30%, 40%, 50%, 60%, 70%, 80%, 90%, 95%, 97%, 98% or 99% of the stable oxygen isotope relative to an unlabeled sample.

Activation reagents for use in the kits of the invention include all of the activation reagents described in the “Labeling” section above. Reagents for donating an amine group for use in the kits of the invention include all such reagents described in the “Labeling” section above.

The kits of the invention can optionally include a trapping reagent that reacts with activated phosphoryl groups to form an isolatable product. The trapping reagent is preferably used when labeling phosphoryl groups at one or two oxygen atom positions and can also be used when labeling all three oxygen atom positions. Trapping reagents include molecules, solution phase reagents, or solid phase reagents that have primary or secondary amine groups for the formation of phosphoramidates or phosphorodiamidates. Trapping reagents also include compounds that can form active esters with phosphate groups.

The kits of the invention optionally can be packaged with purification devices and purification material, or can be used in combination with other commercially available purification devices and purification materials. For example, phosphopeptide samples are first protected at their carboxylate groups on other parts of the molecules, then activated by a carbodiimide reagent, and then purified by a micro-column that is packed with reversed-phase materials. The purified reaction products (i.e., the phosphoramidates or phosphorodiamidates) are then subjected to acid-catalyzed hydrolysis to produce phosphopeptides (or other phosphomolecules) having one, two, or three oxygen isotope labels.

The kits can contain additional reagents to facilitate the labeling or purification of phosphorylated molecules, or can be used in combination with other reagents to facilitate the labeling or purification of phosphorylated molecules. Examples of these reagents include those used to methylate carboxylate groups on phosphorylated molecules, and solvents for purification.

### Determination of Phosphorylation Number of Peptides and Other Biomolecules.

Differential ¹⁸O₁ and ¹⁶O₁ labeling of peptidyl phosphates result in 2 Da difference for each substituted phosphate group, relative to the unsubstituted group. This 2 Da molecular mass difference can be used to determine the phosphorylation number of a molecule in a sample by analyzing a 1:1 mixture of H₂¹⁸O:H₂¹⁶O₁-labeled molecules using mass spectrometry. For example, when peptidyl phosphates are differentially labeled with ¹⁸O₁ versus ¹⁶O₁, monophosphopeptides display a 2 Da mass difference, diphosphopeptides display a 4 Da mass difference, and triphosphopeptides display a 6 Da mass difference. Analogously, when peptidyl phosphates are differentially labeled either with ¹⁸O₁ versus ¹⁷O₁ or with ¹⁷O₁ versus ¹⁶O₁, monophosphopeptides display a 1 Da mass difference, diphosphopeptides display a 2 Da mass difference, and triphosphopeptides display a 3 Da mass difference.

### Multiplexed Quantitation of Phosphopeptides Using Modification-Specific Marker Ions and Tandem Parallel Fragmentation Mass Spectrometry.

The ability to perform multiplex analysis for mass spectrometry analytical methods is highly desirable. In the present invention, up to four samples containing a given phosphomolecule can be differentially labeled on phosphate by exchanging either 0, 1, 2, or 3 atoms of ¹⁸O into each phosphoryl group. Upon pooling the samples, differentially-labeled phosphomolecules can be simultaneously measured by mass spectrometry. The differentially-labeled phosphomolecules differ in mass from one another by 2 Da. The relative mass spectrometric intensities of the differentially-labeled phosphmolecules can be used to calculate the relative concentrations of the same phosphomolecule in four different samples. Both intact large phosphopeptide fragment ions and phosphate-specific ions can be used for the quantitation. The preferred method for generating phosphate-specific ions is parallel fragmentation. The mass range of these peptides spans 6 Da. It is difficult to sample all of the ions as precursors for fragmentation as in standard MS/MS experiments. Parallel fragmentation, without precursor selection, authentically relays the quantitative information in intact peptide ions to phosphate-specific fragment ions. Tandem parallel fragmentation via collision-induced dissociation (CID), or p²CID, has an added advantage of the increased intensities of fragment ions, because virtually all of the gaseous ions of a peptide are sampled for detection (Ramos et al., supra). The principle of p²CID mass spectrometry is the energy-resolved CID of peptidyl (and their fragment) ions of different sizes and charges in the source and collision cell regions.

The principle of multiplexed quantitation by exploiting the differential oxygen labeling methods of the invention combined with tandem parallel fragmentation mass spectrometry analysis is illustrated using a monophosphotyrosinyl (pY) peptide as an example. When the phosphate group on a monophosphotyrosinyl peptide is labeled with 0, 1, 2, or 3 atoms of ¹⁸O, the characteristic immonium ion of pY will change its mass accordingly to be m/z 216, 218, 220, or 222. The relative intensities of these ¹⁸O-substituted immonium ions can be used to measure the relative concentrations of the original phosphopeptide in four different samples. Furthermore, the labile nature of phosphates on phosphoserine (pS) and phosophothreonine (pT) peptides makes their phosphate-specific marker ions better choices than intact ions for sensitive quantitation.

Samples that are combined for multiplexed analysis by mass spectrometry using the oxygen isotope labeling methods of the invention can be identical or different from one another. By way of example, identical multiplexed samples could be from multiple samples of cells that received the same treatment (e.g., an experiment performed in triplicate). An example of different multiplexed samples could be from differentially treated cells, such as a negative control, a positive control, and experimentally treated cells. Another example of different multiplexed samples could be phosphopeptides isolated from brain cells versus phosphopeptides isolated from liver cells. One of ordinary skill in the art will be well aware of how to conduct a multiplexed experiment using the methods of the present invention. For one example of a multiplexed protein quantitation experiment using isobaric tagging reagents, see Ross et al., Mol. Cell. Proteomics 2004, 3, (12), 1154-1169.

### Sample Preparation.

Samples that contain phosphorylated molecules to be analyzed (e.g. but not limited to phosphoproteins) can be obtained from blood, tissue, organ, or cell samples. Tissue samples from human, animal, or man-made organs are homogenized to obtain samples containing phosphorylated molecules of interest, after which the molecules of interest can be further purified, e.g., using reversed-phase liquid chromatography or other appropriate purification methods, as will be understood by one of ordinary skill in the art. Similarly, phosphorylated molecules from clinical plasma samples can be obtained by reversed-phase liquid chromatography or other appropriate purification method.

Cell samples having or potentially having different phosphorylation states can be obtained by stimulating cells with synthetic or naturally-occurring molecules (e.g., pharmaceutical compounds, hormones, growth factors, and the like) or by otherwise changing cell culture conditions (e.g., by changing the growth temperature, subjecting the cells to a mechanical force, placing the cells under oxidative stress, etc.) as will be understood by one of ordinary skill in the art. After the appropriate treatment, cell samples having or potentially having differential phosphorylation states can be lysed to obtain the phosphorylated molecules to be analyzed. See, e.g., Gerber et al., Proc Natl Acad Sci USA 2003, 100, (12), 6940-5.

For example, to analyze phosphoproteins, protein mixtures are subjected to proteolysis to produce peptide mixtures that contain phosphopeptides. The peptide mixtures are desalted and then subjected to oxygen isotopic substitution as described herein. Samples can be differentially labeled with oxygen isotopes (e.g., with 0, 1, 2, or 3 substitutions of phosphoryl oxygen atoms with ¹⁶O, ¹⁷O, or ¹⁸O) and combined together for further purification or for direct mass spectrometric analysis.

### Marker Ions.

The invention provides labeled marker ions that are produced in gas phase, e.g., during mass spectrometer analysis: Po₂⁻, PO₃⁻, HPO₃⁻, H₂PO₄⁻ and immonium ion of phosphotyrosine that have one, two, or three oxygen atoms labeled by a stable oxygen isotope (¹⁶O, ¹⁷O, or ¹⁸O). Such labeled marker ions are produced by subjecting a phosphomolecule labeled with a stable oxygen isotope to a mass spectrometry method such as collision induced dissociation (CID) to detect, identify, quantitate, or analyze the phosphomolecule. By way of example, the skilled artisan understands that CID and other types of gas-phase fragmentation of serine-, threonine-, and tyrosine-phosphorylated peptides under negative ion conditions results in the formation of phosphopeptide-specific marker ions at m/z 79 (PO₃⁻) and m/z 63 (PO₂⁻). Under positive ion conditions serine- and threonine-phosphorylated peptides undergo loss of 98 Da (H₃PO₄) and 80 Da (HPO₃⁻) from the molecular ion, whereas tyrosine-phosphorylated peptides preferentially lose 80 Da. These characteristic fragmentations in the positive and negative ion modes are signatures for phosphopeptides.

### LC-p²CID-MS for Phosphopeptides and Other Phosphorylated Molecules.

In liquid chromatography/tandem parallel collision-induced mass spectrometry (LC-p²CID-MS) experiments, all of the liquid chromatography (LC) elutes are fragmented on an unbiased basis, i.e., no particular precursor selection. Without any selection of particular ions, the quantitative information of phosphopeptides is authentically represented by phosphate-specific ions. The assignment of phosphate-specific ions to particular phosphopeptides can be done after data acquisition; intact ions and fragment ions of the same phosphopeptide have the same elution time (see, e.g., Silva, J. C. et al., Absolute quantification of proteins by LCMSE: a virtue of parallel MS acquisition. Mol Cell Proteomics 2006, 5, (1), 144-56; Steen, H et al., Quadrupole time-of-flight versus triple-quadrupole mass spectrometry for the determination of phosphopeptides by precursor ion scanning. Journal of Mass Spectrometry 2001, 36, (7), 782-790).

In standard LC-MS/MS experiments that use the detection of phosphate-specific marker ions to initiate CID, there is a limitation. Before an LC peak passes, the correct precursor ion that is responsible for the generation of a marker ion may not be “on-the-flight” selected for CID. This is partially due to low concentrations of phosphopeptides (and broadly speaking modified peptides) and partially due to low ionization efficiencies of modified peptides like phosphopeptides. The phosphopeptides often are not the first choices for precursor selection, compared to those co-eluting peptides with relatively high abundance and high ionization efficiency. The present invention overcomes this limitation by providing phosphate-specific marker ions for measuring the levels and phosphorylation states of the intact phosphopeptides.

### Software for Mass Spectrometry.

The invention provides principles and marker ions for constructing instrument-controlling software that allow intelligent data acquisition and thus the detection of phosphorylated molecules, based on the observation of the marker ions with natural oxygen (i.e., ¹⁶O) abundance plus at least another marker ion. The substituted marker ion contains one, two, or three oxygen atoms that are substituted with oxygen isotope ¹⁸O or ¹⁷O. For example, mass spectrometer instrument-controlling software can be designed in such way that when these ions are observed simultaneously, the instrument-controlling software triggers the search for the precursor ions that generate these differentially labeled ions. These precursor ions are then be targeted for fragmentation to obtain structural information. Previously similar methods have been practiced routinely in tandem mass spectrometric analysis of phosphopeptides, based on the observation of individual marker ions that have natural oxygen isotope abundance. The added phosphate-specific marker ions improve the selectivity of the triggering event. The present invention can be used to minimize the observation of “false” phosphorylated molecules in mass spectrometry analysis of mixtures. In this invention, triggering the search for targeted precursor fragmentation only occurs when a conventional marker ion (e.g., a marker ion having only ¹⁶O atoms) is accompanied by at least one substituted marker ion, i.e., a marker ion in which one, two, or three oxygen positions have been substituted with a stable oxygen isotope such as ¹⁸O or ¹⁷O. The labeled marker ions can also be used independently.

The invention also provides principles and marker ions for constructing instrument-controlling software that uses the observation of neutral loss of H₃PO₄ or HPO₃, with one or two oxygen atoms that have enriched oxygen isotopes like ¹⁸O, from phosphorylated molecules to trigger the search for targeted fragmentation of precursor molecules for the observed neutral loss. The neutral loss of molecular fragments stated above can be used independently, or in combination with the observation of neutral loss of H₃PO₄ or HPO₃ with natural oxygen abundance, or with three oxygen atoms labeled with ¹⁸O. Use of neutral loss of H₃PO₄ or HPO₃ with natural oxygen abundance or with all three oxygen atoms labeled with ¹⁸O has been previously described (Zhou et al., Analytical Chemistry 2007, 79, (20), 7603-7610).

The invention further provides principles and marker ions for constructing data processing software for specific analysis of these labeled marker ions and for using these ions for identification and quantitation of phosphopeptides and other phosphorylated molecules. Data processing software can allow extraction of intact and fragment phosphopeptide ions that have the same elution time as the labeled marker ions. The relative intensities of the differentially labeled phosphate-specific marker ions can be used to quantify their precursor molecules, independent of chemical structure variations in other part of molecules. Taking phosphopeptides as examples, different phosphopeptides can have different amino sequences, and thus different natural isotope distributions. When only phosphate-specific marker ions with differential oxygen labels are used, variations in the natural isotope distributions can be corrected consistently. Thus the quantitation based on marker ions with differential oxygen labels are sequence-independent.

### SPECIFIC EXAMPLE

The invention will be described in greater detail by way of a specific example. The following example is offered for illustrative purposes, and is intended to neither limit nor define the invention in any manner.

### Example I

### Oxygen Isotopic Substitution of Peptidyl Phosphates for Phosphate Modification-Specific Mass Spectrometry

### Materials and Methods.

### Reagents and Equipment.

Phosphopeptides were purchased from AnaSpec (San Jose, Calif.). NPF phosphopeptide has a sequence of KRSpYEEHIP (SEQ ID NO: 1); tyrosine protein kinase JAK 2 phosphopeptide has a sequence of VLPQDKEpYpYKVKEPGE (SEQ ID NO: 2); kinase domain of insulin receptor (5) phosphopeptide has a sequence of TRDIpYETDpYpYRK (SEQ ID NO: 3). Reagents were purchased from Sigma-Aldrich (St. Louis, Mo.), Fisher (Pittsburgh, Pa.), Fluka (Milwaukee, Wis.), Acros Organics (Fairlawn, N.J.) or Pierce (Rockford, Ill.) at better than reagent grade. Water-¹⁸O (>95% atom) was purchased from Sigma and Water-¹⁸O (>97% atom) was purchased from Spectra Stable Isotopes (Columbia, Md.) and Cambridge Isotope Laboratories (Andover, Mass.). De-ionized water was purified by a Direct-Q water purifying system (Millipore, Billerica, Mass.). Drying of samples was performed either on SpeedVac (Savant, Farmingdale, N.Y.) or on a lyopholyzer (Labconco, Kansas City, Mo.). High performance liquid chromatography was performed on 10ADvp system (Shimadzu, Columbia, Md.). Mass spectrometry was performed on a quadrupole-time-of-flight tandem mass spectrometer (QTOFmicro, Waters, Milford, Mass.), equipped with an in-house modified electrospray source.

### Methylation of Peptidyl Carboxylates.

Dried phosphopeptides (10 nmol) were incubated with 20 μL of anhydrous methanol at 37° C. for 1 hr. After cooling the peptide solution to 15° C., 80 μL of methanolic HCl (2.5 M, prepared by adding 200 μL of chlorotrimethylsilane to 432 μL of anhydrous methanol and incubating on ice for 10 min) was added. The reaction mixtures were incubated at 15° C. for 1 hr and dried.

### Amidation of Peptidylphosphates.

To dried methylated phosphopeptides (prepared from 10 nmol of phosphopeptides), 10 μL of dimethylsulfoxide was added, followed by the addition of a solution of 20 μL of imidazole solution (1 M, pH 6.0), 7 μL of concentrated HCl and 5 μL of propargylamine. A solution of 1-(3-dimethylaminopropyl)-3-ethylcarbodiimide (EDC) hydrochloride (8 μL of 1.25 M in 1 M imidazole solution at pH 6.0) was further added. The reaction mixtures were incubated at 25° C. for 4 hrs, and then worked up using HLB materials (Waters, Milford, Mass.) that was packed in-house in TopTips (10-200 μL, Glygen, Columbia, Md.). Trifluoroacetic acid (TFA, 0.1% v/v) was used as equilibration and washing solution and 70% (v/v) acetonitrile (ACN) as elution solution. The purified peptides were dried.

### ¹⁸O₁- and ¹⁶O₁-Labeling of Peptidyl Phosphates.

A typical experiment is done by the following protocol. Freeze-dried propargyl phosphoramidate of NPF phosphopeptide (1.2 nmol) was dissolved with 50 μL of 1% (v/v) TFA in H₂¹⁸O and incubated at room temperature for 4 hrs. The reaction mixture was dried and the product was analyzed by mass spectrometry. ¹⁶O₁-labeling of peptidyl phosphates was performed using H₂¹⁶O for hydrolysis.

### Kinetic Study of Hydrolysis of NPF Propargyl Phosphoramidate.

Into 200 μL of H₂¹⁶O, 10 μL of NPF propargyl phosphoramidate (2.36 nmol) in H₂¹⁶O and 5 μL of NPF peptidyl phosphate-¹⁸O₁ (equivalent to a preparation from 236 pmol of NPF phosphopeptide) in H₂¹⁶O were added. An aliquot of 16 μL of the above solution was sampled for liquid chromatography-mass spectrometry (LC-MS) analysis. The remaining solution was added with 1 μL of TFA. The reaction was allowed at 15.0±0.2° C. and the reaction solution was sampled for LC-MS analysis at different time intervals. A control experiment for the NPF peptidyl phosphate-¹⁸O₁ was done as follows: into 194 μL of H₂ ¹⁶O, a solution of 5 μL of NPF peptidyl phosphate-¹⁸O₁, (equivalent to a preparation from 236 pmol of NPF phosphopeptide) in H₂¹⁶O was added, followed by the addition of 1 μL of TFA. The reaction was allowed at 15.0±0.2° C. and the reaction solution was sampled for LC-MS analysis at different time intervals.

The reaction mixture was separated on a reversed-phase column (Atlantis dC₁₈, 3 μm, 1.0×50 mm, Waters, Milford, Mass.). Separation was run at 150 μL/min with a binary gradient [Solvent A: FA:ACN:H₂O=2:10:988 (v/v/v); Solvent B: FA:H₂O:ACN=2:10:988 (v/v/v)]: 8% B at 0 min→8% B at 1 min→12% B at 2 min→18% B at 5.5 min→60% B at 7 min→8% B at 7.1 min→8% B at 8 min. Column temperature was 60° C. Key instrument parameters for mass spectrometer are: capillary voltage 3500 V, cone voltage 28 V, extraction voltage 5 V, collision energy 5 eV, quad set mass 641 m/z and gas cell 300 V.

### Stability Studies of NPF Peptidyl Phosphate-¹⁸O₁.

The ¹⁸O₁-labeled peptidyl phosphate made from NPF phosphopeptide (1.2 nmol) was re-dissolved with 26 μL of H₂¹⁶O and 14 μL of ACN. This solution was divided into 4 aliquots (10 μL each) and the aliquots were added with 60 μL of 0.1% (v/v) TFA (giving a final pH of 2.04), 60 μL of 0.2% (v/v) formic acid (FA; giving a final pH of 2.56), 60 μL of ammonium acetate (58 mM; giving a final pH of 6.21) and 60 μL of ammonium bicarbonate (58 mM; giving a final pH of 8.45), respectively. These solutions were incubated at 15.0±0.2° C. and analyzed by liquid chromatography mass spectrometry (LC-MS) at different time intervals, using the same LC and MS conditions as those above.

### Mass Spectrometric Counting of the Phosphorylation Number of Phosphopeptides.

Peptidyl phosphate-¹⁶O₁ and phosphate-¹⁸O₁, labeled separately, were mixed to obtain roughly equal intensities for the monoisotopic peaks of the phosphate-¹⁶O₁ and phosphate-¹⁸O₁, respectively. Mass spectra were acquired using standard parameters.

### Tandem Parallel Fragmentation Mass Spectrometry of Peptidyl Phosphate-¹⁸O₁/¹⁶O Pairs.

Peptidyl phosphate-¹⁶O₁ and phosphate-¹⁸O₁, labeled in parallel from two aliquots of phosphopeptides, were mixed at different volume ratios and analyzed by LC-p²CID mass spectrometry. LC conditions were similar to the above. Key mass spectrometer parameters for p²CID experiments were: capillary voltage 3500 V, cone voltage 40 V, extraction voltage 5 V, collision energy 52 eV, quad set mass m/z 400, and gas cell 300 V.

### Results and Discussion

### Principle for Oxygen Isotopic Substitution of Peptidyl Phosphates.

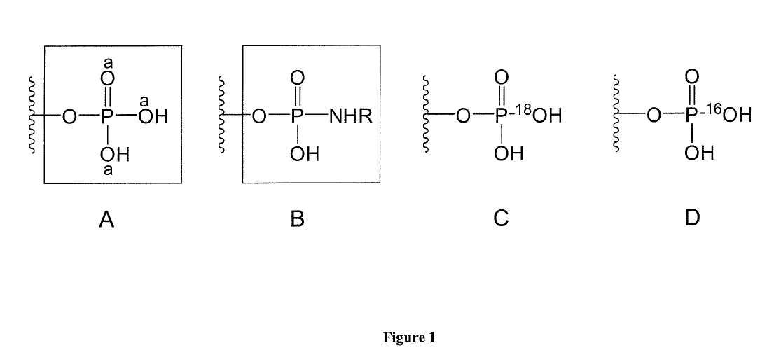

The direct labeling of peptidyl phosphate oxygen causes no other alterations in peptide structure, except for one atom of nonbridging oxygen (FIG. 1). Without any addition of other atoms to the phosphate, this stable isotope substitution strategy exhibited the highest atom labeling efficiency. The most important advantage of the strategy is that it allows the straight translation of modification-specific mass spectrometry (Carr et al., Methods Enzymol 2005, 405, 82-115), the mass spectrometric methods that are specifically developed toward particular protein modifications like protein phosphorylation. The principle of isotopic substitution has been practiced for quantitative analysis of proteins and proteomes. Examples include proteins that are labeled at amino acid residues and peptides that are labeled at C-termini by enzymatic reactions and by microwave-assisted acid hydrolysis, as well as kinase-catalyzed in vitro labeling of proteins using ATP-γ-¹⁸O₄ as co-substrate. However, labeling of the pre-existing phosphates on polypeptides has not been achieved.

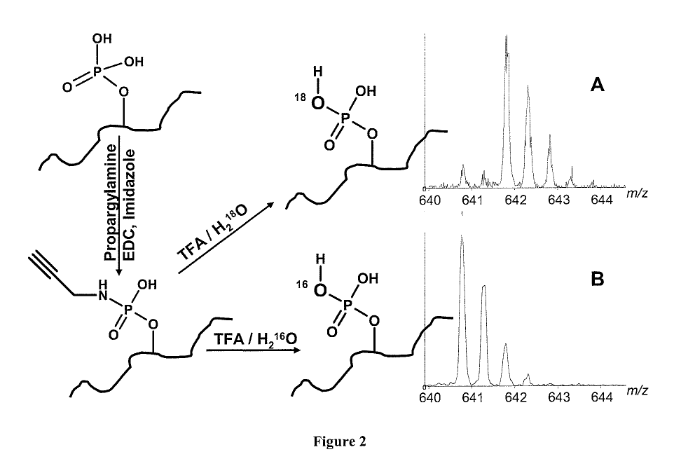

Carbodiimides assist the formation of phosphoramidates from peptidyl phosphates and the phosphoramidates can be hydrolyzed with acid-catalysis to regenerate phosphates with the defined oxygen isotope substitution (FIG. 2). When this reaction is performed in H₂¹⁸O solution, the regenerated phosphate group incorporates one ¹⁸O atom to form peptidyl phosphate-¹⁸O₁, and produces a mass increase by 2.004 Da compared to that in H₂¹⁶O solution (FIG. 2). When this chemical mediation strategy of oxygen exchange on phosphates is performed in parallel for two phosphopeptide sample pools, using H₂¹⁶O and H₂¹⁸O respectively, phosphopeptide signals show up in pairs in mass spectra. Phosphopeptides with one phosphate group are separated by 2.004 Da, i.e., the mass difference between one atom of ¹⁶O and ¹⁸O; phosphopeptides with two phosphate groups by 4.008 Da; phosphopeptides with three phosphate groups by 6.012 Da.

### Implementation of Oxygen Labeling of Peptidyl Phosphates by Carbodiimide-Assisted Amidation and Acid-Catalyzed Hydrolysis.

Phosphopeptides were methylated with minimal partial reactions, using methanolic HCl to protect carboxylate groups on peptides from the consequent carbodiimide-assisted amidation reaction. Dissolving chlorotrimethylsilane in anhydrous methanol provided a convenient way for preparing methanolic HCl with defined HCl concentrations. The methylated phosphopeptides were amidated with propargylamine using EDC (FIG. 2). Propargylamine was found to be an efficient amidation reagent for peptidyl phosphates, compared to cystamine.

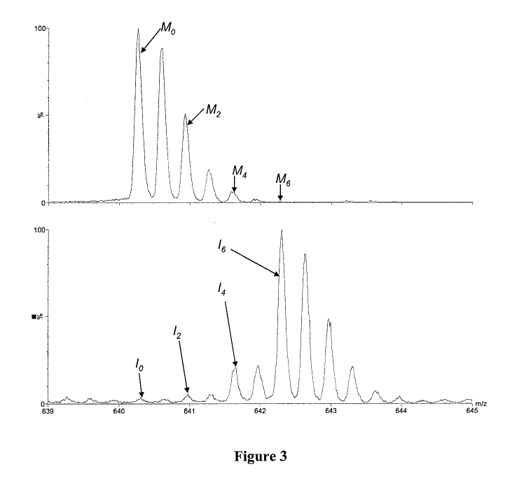

Acid-catalyzed hydrolysis of the newly generated phosphoramidates, using 1% (v/v) TFA in H₂¹⁸O resulted in the incorporation of one, and only one, ¹⁸O atom in each peptidyl phosphate. The residual phosphate-¹⁶O₁ in the ¹⁸O₁-labeled samples is observed; it can be quantified by mass spectrometry, according to Equation 1. The amount of phosphate-¹⁸O₁ can be calculated according to Equation 2.

$$\begin{matrix}{[ Phosphate - {}_{ }^{16}{O}{}_{1}^{ } ]}^{{H}_{2}^{18} O} = {\sum }_{i = 0}^{n} ( n - i ) \times {x}_{i} & Equation 1 \\ {[ Phosphate - {}_{ }^{18}{O}{}_{1}^{ } ]}^{{H}_{2}^{18} O} = {\sum }_{i = 0}^{n} i \times {x}_{i} & Equation 2\end{matrix}$$

where xᵢ is the concentration of a phosphopeptide that has n numbers of phosphates and i of them are labeled with ¹⁸O₁, i.e., (n−i) of them are labeled with ¹⁶O₁. For example, for a phosphopeptide with three phosphates (FIG. 3), i.e., n=3, the ratio of the residual phosphate-¹⁶O₁ to the phosphate-¹⁸O₁ can be calculated according to Equation 3:

$${r}_{0} = \frac{{[ Phosphate {}_{ }^{16}{O}{}_{1}^{ } ]}^{{H}_{2}^{18} O}}{{[ Phosphate {}_{ }^{18}{O}{}_{1}^{ } ]}^{{H}_{2}^{18} O}} = \frac{3 {x}_{0} + 2 {x}_{1} + {x}_{2}}{{x}_{1} + 2 {x}_{2} + 3 {x}_{3}} = \frac{3 {I}_{0} + 2 ( {I}_{2} - {I}_{0} {M}_{2} ) + [ {I}_{4} - {I}_{0} {M}_{4} - {M}_{2} ( {I}_{2} - {I}_{0} {M}_{2} ) ]}{\begin{matrix}( {I}_{2} - {I}_{0} {M}_{2} ) + 2 [ {I}_{4} - {I}_{0} {M}_{4} - {M}_{2} ( {I}_{2} - {I}_{0} {M}_{2} ) ] + \\ 3 \{ {I}_{6} - {I}_{0} {M}_{6} - {M}_{4} ( {I}_{2} - {I}_{0} {M}_{2} ) - \\ {M}_{2} [ {I}_{4} - {I}_{0} {M}_{4} - {M}_{2} ( {I}_{2} - {I}_{0} {M}_{2} ) ] \}\end{matrix}} \qquad Equation 3$$

where I₀ is the observed intensity at the mass-to-charge ratio that is equal to that of the monoisotopic peak of an ¹⁶O₁-labeled species, I₂ is the intensity at 2 Da higher, I₄ is at 4 Da higher, and I₆ is at 6 Da higher; M₀ is the natural abundance of the monoisotopic peak of a species, M₂ is 2 Da higher, M₄ is 4 Da higher, and M₆ is 6 Da higher. The M values can be either experimentally determined (on a QqTOF instrument) or theoretically calculated; the corresponding values are very similar. For the permethylated insulin receptor phosphopeptide (3 phosphates), M₂ is 0.5145, M₄ is 0.0642, and M₆ is 0.0040 (theoretical). The value of ro for an ¹⁸O₁-labeled sample was calculated to be 0.122 (i.e. 10.9% of residual phosphate-¹⁶O₁). The residual phosphate-¹⁶O₁ for the ¹⁸O₁-labeled samples of NPF peptide (1 phosphate) and JAK 2 peptide (2 phosphates) can be calculated similarly. In general, the observed residual amounts (about 5˜20%) of phosphate-¹⁶O₁ in the ¹⁸O₁-labeled samples are higher than the expected theoretical values that are calculated based on the ¹⁸O atom enrichment of water used. The residual phosphate-¹⁶O₁ could be attributed to the residual H₂¹⁶O in H₂¹⁸O used as well as in the dry phosphoramidate, the unreacted NPF phosphate-¹⁶O₁ during the EDC-assisted amidation and the hydrolysis product during the workup of phosphoramidates. The overall recoveries for the oxygen isotopic substitution of phosphate groups on permethylated phosphopeptides (see experimental; steps include EDC-assisted amidation, microscale workup and acid-catalyzed hydrolysis) can be readily determined by mass spectrometric quantitation of mixtures of reactants (peptidyl phosphates-¹⁶O₁) and products (peptidyl phosphates-¹⁸O₁). The overall recoveries are routinely >45%.

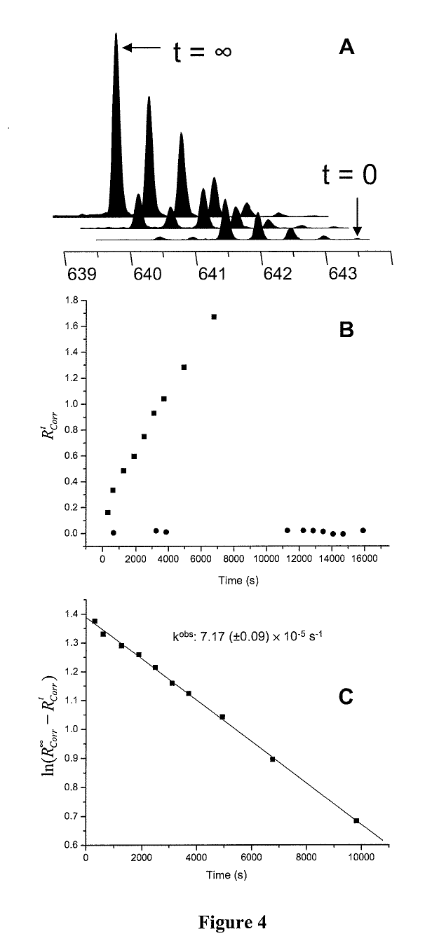

The hydrolysis kinetics of the phosphoramidate of the NPF peptide was further studied in detail in 0.5% (v/v) TFA solution. The NPF peptidyl phosphate-¹⁸O₁ is used as the internal standard (FIG. 4A at t=0); it does not exchange the ¹⁸O atom with water in the TFA solution and thus has constant concentration (FIG. 4B). The hydrolysis reaction follows pseudo first-order reaction kinetics with an observed rate constant of 7.17(±0.09)×10⁻⁵ s⁻¹ that is the slope of a linear plot of ln Q versus time (FIG. 4C). Q is calculated according to Equation 4:

$$Q = {\{ \frac{( {I}_{0} - {r}_{0} {I}_{2} ) / ( 1 - {M}_{2} {r}_{0} )}{( 1 + {r}_{0} ) ( {I}_{2} - {M}_{2} {I}_{0} ) / ( 1 - {M}_{2} {r}_{0} )} \}}^{∞} - {\{ \frac{( {I}_{0} - {r}_{0} {I}_{2} ) / ( 1 - {M}_{2} {r}_{0} )}{( 1 + {r}_{0} ) ( {I}_{2} - {M}_{2} {I}_{0} ) / ( 1 - {M}_{2} {r}_{0} )} \}}^{t} \qquad Equation 4$$

where the variables are defined as in Equation 3. For the permethylated NPF phosphopeptide, M₂ is experimentally measured to be 0.2538 (the theoretical value is 0.2664).
Sequence-Independent Quantitation Using ¹⁶O₁/¹⁸O₁-Labeled Phosphate-Specific Marker Ions and Tandem Parallel Collision-Induced Dissociation Mass Spectrometry (p²CID-MS).

The phosphate-specific marker ions are independent from the amino acid sequences of phosphopeptides and have unique mass defect that is originated from phosphorus element. These ion characteristics provide a unique opportunity for developing a method of sequence-independent quantitation of phosphopeptides.

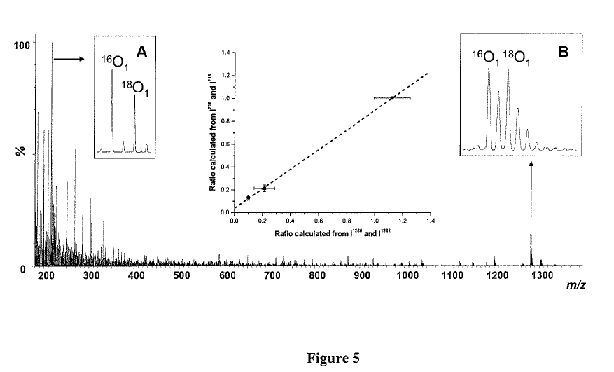

In this work, pY peptides are used as models; they characteristically produce positive immonium ion of pY at m/z 216.043. When pY peptides with differential ¹⁶O₁ and ¹⁸O₁ labels are dissociated via collision-induced dissociation (CID), the immonium ions show in pair at m/z 216.043 and 218.047. Their intensities can be used for relative quantitation of the pY phosphopeptides with different isotope labels (Insert A in FIG. 5), without the need of sequence-dependent deconvolution of the isotope distribution of peptidyl ions. The pY immonium ion has a defined isotope distribution. Using a method of p²CID-MS (Ramos et al., Anal. Chem. 2006, 78, (18), 6391-6397) that sequentially combines the parallel CID (Purvine et al., Proteomics 2003, 3, (6), 847-850) in the source of mass spectrometers (Purvine, supra; Loo et al., Rapid Commun Mass Spec 1988, 2, (10), 207-10; Zhai et al., Anal Chem 2005, 77, (18), 5777-84; Van Dongen et al., Rapid Commun Mass Spec 1999, 13, (17), 1712-1716) and the parallel CID in the collision cell, both the pY immonium ions (Insert A in FIG. 5) and singly charge molecular ions of phosphopeptides (Insert B in FIG. 5) can be simultaneously observed in a single experiment (FIG. 5). An advantage of the p²CID method is increased sensitivity for fragment ions, because virtually all of the peptidyl ions of different charge states and of different degrees of intactness that are generated during ionization are sampled to produce fragment ions, e.g., the 216.043 and 218.047 marker ions. Without precursor selection in p²CID-MS experiments, the quantitative information in the differentially labeled peptides can be authentically sampled to produce fragment ions for quantitative measurements of phosphopeptides (FIG. 5). In contrast, in standard MS/MS experiments the precursors of differentially labeled peptides need to be concurrently selected to produce fragment ions. Such a selection could be difficult for phosphopeptides with more than three phosphates; the precursor selection window needs to pass all of the peptidyl ions that are separated by more than 6.012 Da. It should be noted that the use of p²CID-generated phosphate-specific marker ions for quantitation requires (at least partially) separated phosphopeptides (e.g., by liquid chromatography as in this work) so that the relative intensity of ¹⁶O₁ and ¹⁸O₁-labeled marker ions can be correlated to particular precursor phosphopeptides in a mixture.

The concentration ratio of the peptidyl phosphate in the ¹⁶O₁-labeling pool to that in the ¹⁸O₁-labeling can be calculated according to Equation 5:

$$\frac{{[ Phosphate ]}^{{H}_{2}^{16} O}}{{[ Phosphate ]}^{{H}_{2}^{18} O}} = \frac{( 1 + {r}_{0} {M}_{2} ) {I}_{0} - {r}_{0} {I}_{2}}{( 1 + {r}_{0} ) {I}_{2} - {M}_{2} ( 1 + {r}_{0} ) {I}_{0}} \qquad Equation 5$$

where the variables are defined as in Equation 3. For the pY immonium ion, M₂ is 1.23%. The molecular ions for the differentially labeled NPF peptides are also used to calculate the relative concentration of the phosphopeptides in the two sample pools, according to Equation 5. These two sets of data are in good agreement with each other as in the inserted plot in FIG. 5, indicating that p²CID-MS produces phosphate-specific marker ions with the same quantitative information as that of the intact ions. The measurements based on the singly-charged molecular ions appear to have larger errors. This could reflect the fact that these measurements are related to a small difference between two large numbers (Equation 5). The first is the observed intensity of the 12 peak and the second is the M₂I₀ (M₂=0.2538) value, the M₂ contribution from the peptidyl phosphate-¹⁶O₁ to the observed intensity of the I₂ peak (Insert B in FIG. 5). Therefore, the precision of the measurements is heavily dependent on the capability of a mass spectrometer measuring accurate isotopic distributions. Inverse labeling experiments could provide a solution to this problem. In contrast, the M₂ (0.0123) of ¹⁶O₁-labeled pY immonium ion contributes minimally to the peak at m/z 218.047, the monoisotopic peak of the ¹⁸O₁-labeled pY immonium ion.

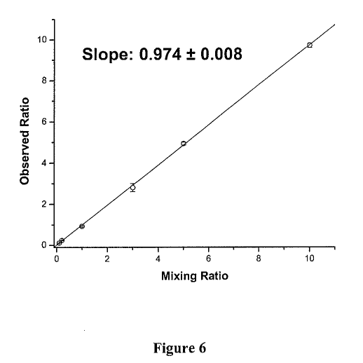

Attributing to this nature of pY immonium ions, the relative phosphopeptide quantitation, using the m/z 216.043 and 218.047 marker ions from p²CID-MS, has a dynamic range at least two orders of magnitude (FIG. 6). The plot of the observed relative concentrations of phosphopeptides in two sample pools against the theoretical ones has a near to unity slope. A limitation of using small marker ions for quantitation could come from the chemical noise generated by fragmentation processes, but this is a much less issue when high-resolution mass spectrometers are used that can take advantage of the unique mass defect of ³¹Pₚ for improving the quantitation specificity.

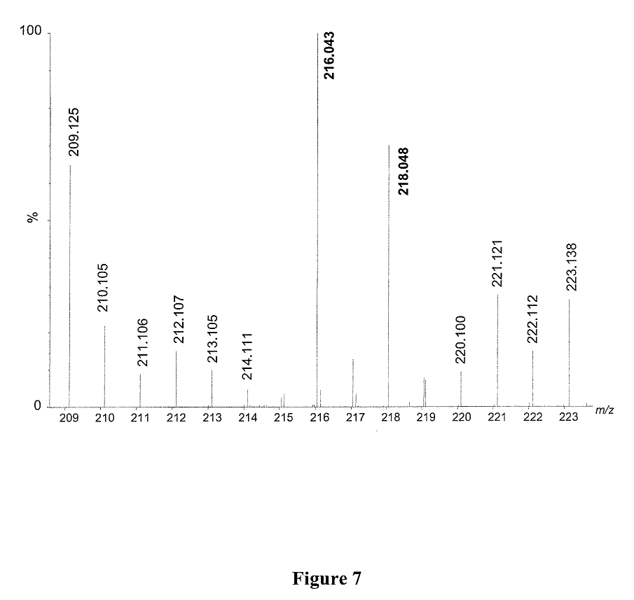

Phosphorylation of a hydroxyl group adds HPO₃ to a peptide, and causes an additional mass defect of −0.03368 Da. This mass difference requires a resolution of about 6500 at m/z 216 to separate the pY immonium ion from isobaric nonphosphoryl ions. Using centroid mass spectra (FIG. 7), the average mass defect for ions with nominal mass-to-charge ratios around 216 (209-213 and 221-223) is calculated to be 0.114±0.011 Da. It is significantly higher than the mass defect of 0.043 Da at m/z 216 for the ¹⁶O₁-labeled pY immonium ion and 0.048 Da at m/z 218 for the ¹⁸O₁-labeled pY immonium ion (FIG. 7). Therefore, for phosphate-specific marker ions, the unique mass defect of phosphorus element promises the ready separation of the phosphate-specific maker ions from isobaric nonphosphoryl fragment ions in mass spectra of high resolution. This establishes a solid basis for phosphopeptide quantitation using phosphate-specific marker ions. In principle, this strategy can be applied to quantify pS and pT peptides using other phosphate-specific marker ions like m/z 78.959 (PO₃⁻) and 96.969 (H₂PO₄⁻).

### Mass Spectrometric Counting of the Phosphorylation Number.

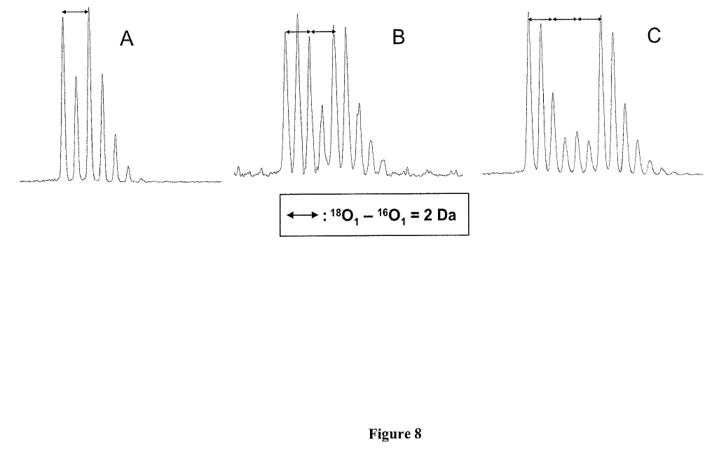

Acid hydrolysis of peptidyl phosphoramidates in H₂¹⁸O resulted in the successful incorporation of one ¹⁸O atom in each peptidyl phosphate on phosphopeptides. The mass difference of 2.004 Da between ¹⁸O and ¹⁶O (or m/z=2.004/1 for singly charged ions, m/z=2.004/2 for doubly charged ions, and m/z=2.004/3 for triply charged ions, etc.) can be used as a molecular ruler to count the number of phosphate groups on phosphopeptides. Doubly charged ions for NPF, JAK2 and insulin phosphopeptides show isotope patterns in doublets that have mass separations equal to 1 time, 2 times, and 3 times of 2.004 Da, respectively (FIG. 8). Therefore, the NPF peptide has 1 phosphate group, the JAK 2 peptide has 2 phosphate groups, and the insulin peptide has 3 phosphate groups. The same counting strategy, in principle, could be applied to fragment ions of phosphopeptides. If the needed sequence ions are generated via CID or electron transfer dissociation (Molina et al., Proc Natl Acad Sci USA 2007, 104, (7), 2199-204), the differential oxygen labels could further help pinpointing phosphorylation sites on peptides (Zhou et al., Anal. Chem. 2007, 79, (20), 7603-7610). There is a discontinuity or change in paired isotope patterns of sequence ions before and after a particular phosphorylation site.

Stability of ¹⁸O-Labeled Peptidyl Phosphates.

Back-exchange reactions of peptidyl phosphate-¹⁸O₁ were examined in 0.1% (v/v) TFA, 0.2% (v/v) FA, 20 mM ammonium acetate (pH 7.0), and 50 mM ammonium bicarbonate (pH 8.0); all of the reaction solutions contained 5% (v/v) acetonitrile to minimize the adsorption loss of peptides during the period of 5 days for the stability studies of the ¹⁸O label. For all of the conditions, the relative ratios of the m/z 642 peak to the 640 peak for the [M+2H]²⁺ ions of ¹⁶O₁- and ¹⁸O₁-labeled NPF peptides were constant, indicating that there were no detectable back exchange of the labeled phosphate oxygen with the water oxygen. The oxygen labels in ¹⁸O₁-labeled NPF peptide has also been found stable in 0.5% (v/v) TFA in overnight experiments. The examined conditions are commonly used during sample preparation and analysis of phosphopeptides. The high stability of the oxygen labels on peptidyl phosphates is essential to the use of the stable isotopic substitution method for quantitative phosphopeptide analysis. These observations also emphasize the importance of chemical mediation for successful oxygen exchange to occur on peptidyl phosphates.

### INCORPORATION BY REFERENCE

Throughout this application, various publications, patents, and/or patent applications are referenced in order to more fully describe the state of the art to which this invention pertains. The disclosures of these publications, patents, and/or patent applications are herein incorporated by reference in their entireties to the same extent as if each independent publication, patent, and/or patent application was specifically and individually indicated to be incorporated by reference.

### OTHER EMBODIMENTS

It will be apparent to those of ordinary skill in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of labeling a phosphomolecule with a stable oxygen isotope, comprising:
a) activating a phosphoryl group covalently bound to the phosphomolecule, and
b) hydrolyzing the activated phosphoryl group with water enriched for a stable oxygen isotope,
thereby labeling the phosphomolecule with a stable oxygen isotope.

2. The method of claim 1, further comprising amidating the activated phosphoryl group prior to hydrolyzing the activated phosphoryl group with water enriched for a stable oxygen isotope.

3. The method of claim 1 wherein the phosphomolecule is a phosphopeptide.

4. The method of claim 1, wherein the stable oxygen isotope is ¹⁶O, ¹⁷O, or ¹⁸O.

5. A kit for labeling a phosphoryl group on a phosphomolecule with a stable oxygen isotope, comprising:
a) a reagent for activating the phosphoryl group covalently bound to the phosphomolecule;
b) a reagent for donating an amine group to form an amide bond with the phosphorus of a phosphate group that is covalently bound to the phosphomolecule; and
c) instructions for using the kit to label a phosphoryl group on a phosphomolecule with a stable oxygen isotope.

6. The kit of claim 5, further comprising water that is enriched for a stable oxygen isotope.

7. The kit of claim 5, further comprising a reagent for methylating carboxyl residues of the phosphomolecule prior to activating the phosphoryl group covalently bound to the phosphomolecule.

8. The kit of claim 5, further comprising a control phosphomolecule.

9. The kit of claim 5, further comprising a material for purifying a labeled phosphomolecule from a reaction mixture.

10. A kit for labeling a phosphoryl group on a phosphomolecule with a stable oxygen isotope, comprising:
a) a reagent for activating the phosphoryl group covalently bound to the phosphomolecule;
b) a reagent for donating a functional group to form an active ester with a non-bridging oxygen of a phosphate group that is covalently bound to the phosphomolecule; and
c) instructions for using the kit to label a phosphoryl group on a phosphomolecule with a stable oxygen isotope.

11. The kit of claim 10, further comprising water that is enriched for a stable oxygen isotope.

12. The kit of claim 10, further comprising a reagent for methylating carboxyl residues of the phosphomolecule prior to activating the phosphoryl group covalently bound to the phosphomolecule.

13. The kit of claim 10, further comprising a control phosphomolecule.

14. The kit of claim 10, further comprising a material for purifying a labeled phosphomolecule from a reaction mixture.

15-27. (canceled)

28. A method of detecting a pre-existing phosphoryl group on a phosphomolecule, comprising:
a) subjecting a phosphomolecule labeled with a stable oxygen isotope to a gas-phase dissociation method to generate a labeled phosphate-specific marker ion, wherein the phosphoryl group has at least one non-bridging oxygen atom position labeled with a stable oxygen isotope; and
b) detecting an ion signal produced by the labeled marker ion, thereby detecting the pre-existing phosphoryl group on the phosphomolecule.

29. The method of claim 28, wherein the phosphoryl group has a bridging oxygen atom position not labeled with a stable oxygen isotope.

30. A method of measuring the number of pre-existing phosphoryl groups on a phosphomolecule in a sample, comprising:
a) subjecting a labeled phosphomolecule from a first sample to a gas-phase dissociation method to generate a labeled phosphate-specific marker ion, wherein the phosphoryl group in the labeled phosphomolecule has at least one non-bridging oxygen atom position labeled with a stable oxygen isotope;
b) measuring an ion signal produced by the labeled phosphate-specific marker ion;
c) comparing the ion signal from the labeled phosphate-specific marker ion from the first sample to an ion signal produced by a labeled phosphate-specific marker ion from the labeled phosphomolecule from a second sample, wherein the labeled phosphomolecule from the second sample has a different number of non-bridging oxygen atom positions labeled with a stable oxygen isotope than does the labeled phosphomolecule from the first sample; and
d) correlating a difference in ion signal between the first sample and the second sample with the number of pre-existing phosphoryl groups on the labeled phosphomolecule,
thereby measuring the number of pre-existing phosphoryl groups on a phosphomolecule in a sample.

31. The method of claim 30, wherein the labeled phosphomolecule from the first and second samples is combined into a single sample prior to subjecting the phosphomolecule to a gas-phase dissociation method.

32. The method of claim 31, further comprising combining the labeled phosphomolecule from a third sample with the labeled phosphomolecule from the first and second samples prior to subjecting the phosphomolecule to a gas-phase dissociation method, comparing ion signals produced from all three samples, and correlating a difference in ion signal among all three samples with the number of pre-existing phosphoryl groups on the labeled phosphomolecule, wherein the labeled phosphomolecule from the third sample has a different number of oxygen atom positions labeled with a stable oxygen isotope than does the labeled phosphomolecule from the first and second samples.

33. The method of claim 32, further comprising combining the labeled phosphomolecule from a fourth sample with the labeled phosphomolecule from the first, second, and third samples prior to subjecting the phosphomolecule to a gas-phase dissociation method, comparing ion signals produced from all four samples, and correlating a difference in ion signal among all four samples with the number of pre-existing phosphoryl groups on the labeled phosphomolecule, wherein the labeled phosphomolecule from the fourth sample has a different number of oxygen atom positions labeled with a stable oxygen isotope than does the labeled phosphomolecule from the first, second, and third samples.

34. The method of claim 30, wherein the phosphoryl group has a bridging oxygen atom position not labeled with a stable oxygen isotope.

35. A method of measuring the relative amounts, in two samples, of a phosphomolecule having a pre-existing phosphoryl group, comprising:
a) combining together a first sample with a second sample to produce a single sample, wherein the phosphoryl group in the first sample is unlabeled and the phosphoryl group in the second sample is labeled with ¹⁷O or ¹⁸O;
b) comparing an ion signal produced by the unlabeled phosphate-specific marker ion with an ion signal produced by the labeled phosphate-specific marker ion; and
c) correlating a difference in the ion signal produced by the unlabeled phosphate-specific marker ion versus the ion signal produced by the labeled phosphate-specific marker ion with the relative amounts, in the two samples, of a phosphomolecule having a pre-existing phosphoryl group,
thereby measuring the relative amounts, in two samples, of a phosphomolecule having a pre-existing phosphoryl group.

36-40. (canceled)
